(19) United States
(12) Patent Application Publication
(10) Pub. No.: US 2008/0255546 A1
(43) Pub. Date: Oct. 16, 2008

(54) Eye Accommodation Recovery

(76) Inventor: Leonid Sergeevich Orbachevski, Moscow (RU)

Correspondence Address: PATENT, COPYRIGHT & TRADEMARK LAW GROUP, 4199 Kinross Lakes Parkway, Suite 275, RICHFIELD, OH, 44286, US

(21) Appl. No.: 12/091,273

(22) PCT Filed: Oct. 24, 2005
(86) PCT No.: PCT/RU2005/000520

§ 371 Date: Apr. 23, 2008

(51) Int. Cl. A61F 9/008

(52) U.S. Cl. 606/4

(57) ABSTRACT

A method for eye accommodation recovery comprises transscleral pulse infrared laser radiation treating ciliary muscle and using the visible light for fixing sight direction, wherein in one alternative of the method color of light used for fixing the sight direction and treating eye retina is altered, hence, controlling the state of the accommodation apparatus of the eye, and pulse infrared laser radiation intensity treating ciliary muscle is changed simultaneously. Another alternative of the method comprises dispersion of visible light in the vicinity of the eye pupil providing dispersed light effect on a greater area of eye retina. Color of visible light is altered simultaneously, hence, controlling the state of the accommodation apparatus of the eye.

### FIELD OF THE INVENTION

An invention relates to methods and devices for eye treatment, comprising methods and devices of eye disorders light therapy applying a coherent laser radiation and an incoherent optical radiation.

### BACKGROUND OF THE INVENTION

There has been known a method for accommodation recovery described in RU Patent 2,169,547 (publication date Jun. 27, 2001), comprising transscleral laser radiation effect on a ciliary muscle. The method comprises a combination of said effect of optical radiation with electrical stimulation on a patient and drug application and is a complex requiring the use of several devices and long-term treatment.

There has been known a method for accommodation recovery described in CA Patent 2,409556 (publication date Apr. 23, 2004), comprising transscleral laser radiation effect on a ciliary muscle and vision stimulation by a light source radiation, used for fixing sight direction. However, in said method vision stimulation causes accommodation apparatus strain, said ciliary muscle, in particular. This counteracts said muscle relaxation by transscleral laser irradiation that limits application of said method.

There has been known a method for accommodation recovery described in RU Patent 2,201,180 (publication date Mar. 27, 2003), comprising a pulse transscleral effect of infrared laser radiation on said ciliary muscle and the use of visible radiation for fixing sight direction. This method is the closest analogue to said methods. A disadvantage of said method is limited (indirect) use of radiation fixing the sight direction as a treatment effect factor.

There have been known devices for therapy applied to ophthalmology described in WO 99/39669 (international publication date Aug. 12, 1999) and WO 99/63916 (international publication date Jun. 11, 1999), each comprising a module for transscleral effect of laser radiation on said ciliary muscle of the eye and visible radiation former for fixing sight direction. Said device application is limited due to nonuse abilities to affect eye retina in full.

There has been known a device for therapy applied to ophthalmology described in RU Patent 2,201,180 (publication date Mar. 27, 2003), comprising at least a single module for transscleral affecting said ciliary muscle of eye by pulse infrared laser radiation and fixing the sight direction, comprising beam former of pulse laser infrared radiation for affecting said ciliary muscle and visible light former. Said device application is limited and provides no ability to treat said eye retina by visible spectrum radiation.

### SUMMARY OF THE INVENTION

The invention comprising two methods of eye accommodation recovery and two devices for therapy applied to ophthalmology is aimed at increasing efficiency of treating accommodation weakness via transscleral effect of infrared laser radiation on eye muscles through additional influence on the accommodation apparatus, via corresponding effect on retina and visual conductive tracts, simultaneously used for fixing the sight direction. The invention provides a combination of two types of influence on the eye accommodation apparatus via affecting eye retina and visual conductive tracts by visible light, also fixing the sight direction, and direct effect on said ciliary muscle by pulse infrared laser radiation. To put it differently, visible light fixing the sight direction is also used for affecting the visual tract in a manner of relaxing or straining the accommodation apparatus. Simultaneous use of infrared laser radiation for transscleral affecting the ciliary muscle (the main part of the accommodation apparatus) amplifies the eye accommodation recovery effect.

Said technical result is obtained in the method for eye accommodation recovery, comprising pulse transscleral effect of infrared laser radiation on the ciliary muscle using visible light for fixing the sight direction. Color and brightness of light used for fixing the sight direction and treating eye retina are changed, hence, controlling the state of the accommodation apparatus of the eye, and intensity of pulse infrared laser radiation affecting the ciliary muscle is varied simultaneously.

As intensity of laser radiation for transscleral treatment of the ciliary muscle is varied, high intensity of laser radiation can be timed up with retina treatment by radiation inducing the highest relaxation of the accommodation apparatus of the eye (ciliary muscle). Regular changes in intensity of laser radiation providing transscleral treatment of the ciliary muscle can be synchronized with retina treatment by light of various color and brightness to reach optimal therapeutic effect.

Treatment light color can be varied smoothly (stepless) in the visible spectrum, and the variation rate in some regions of the visible spectrum and regularity can be selected individually. This allows for activating and stimulating all conductive visual tracks.

In addition, the source of visible light can be drawn nearer to or removed from the eye. Hence, it is natural to change visual angular dimension of the radiation source.

Said technical result is obtained in a method for eye accommodation recovery as well, comprising pulse transscleral effect of infrared laser radiation on the ciliary muscle applying visible light to fix the sight direction, wherein a part of radiation used for fixing the sight direction is dispersed in the immediate vicinity of the eye. Hence, retina of the eye is treated by dispersed visible light with simultaneously altered color and, therefore, the state of the accommodation apparatus of the eye is controlled. We emphasize that contrary to radiation fixing the sight direction, light dispersed in the immediate vicinity of the eye treats the whole retina area. In addition, brightness of treating light can be varied simultaneously with dispersed light effect.

Radiation used for fixing the sight direction can be dispersed regularly. Hence, time period between two treatments of retina by dispersed radiation should be selected so that the eye is unable to resolve the dispersion regularity e.g. below 0.04 s.

Transscleral laser radiation intensity affecting the ciliary muscle can be increased during retina treatment by dispersed radiation. In particular, dispersed light, green, for example, treatment of retina relaxes the accommodation apparatus that has a beneficial effect on laser radiation treatment results directly for the ciliary muscle.

Transscleral treatment of ciliary muscle by infrared laser radiation can be performed regularly, in the time period of retina treatment by dispersed light.

With respect to radiation used for fixing the sight direction, dispersed light color can be altered.

A part of radiation affecting retina can be dispersed permanently.

The ratio of dispersed and collimated (used for fixing the sight direction) components of radiation affecting retina can be changed regularly.

Said technical result is obtained in a device for therapy applied to ophthalmology, comprising at least a single module for pulse transscleral infrared laser radiation treating ciliary muscle of the eye and fixing the sight direction, in turn comprising a former operative to produce the pulse infrared laser radiation and a former operative to produce the visible light. Moreover, the device comprising an additional control module connected to the visible light former operative to vary both light color and intensity, and to the infrared laser radiation former for synchronizing changes in color and intensity of the visible light treating retina and variations in laser radiation intensity for transscleral treatment of the ciliary muscle.

Said device can comprise the second module for transscleral pulse infrared laser radiation treating ciliary muscle of the eye and fixing the sight direction, comprising the former of pulse infrared laser radiation and the former of visible light operative to vary color and intensity of light. Hence, the control module is connected to the formers of visible light and the formers of pulse infrared laser radiation of both modules.

The control module can provide for setting sequences for varying visible light color and intensity treating the eye retina and intensity of transscleral laser radiation treating ciliary muscle of the eye.

Said technical result is obtained in a device for therapy applied to ophthalmology, comprising at least a single module for pulse transscleral treating ciliary muscle of the eye by infrared laser radiation and fixing the sight direction. In addition, the device comprises a control unit; the former of visible light is operative to vary light color and comprises a supplementary component with controllable dispersion of the visible light set at the output pupil of the former. Hence, the control unit is connected to the visible light former and pulse infrared laser radiation former for synchronizing color and dispersion variations of the visible light treating retina of the eye, and intensity variations of transscleral laser radiation treating ciliary muscle of the eye.

Said device can comprise the second module for transscleral pulse infrared laser radiation treating ciliary muscle of the eye and fixing the sight direction, comprising the former of pulse infrared laser radiation and the former of visible light operative to vary color and intensity of light, comprising the component operative to controllable dispersion of the visible light. Hence, the control unit can be connected to the formers of visible light and the formers of pulse infrared laser radiation of both modules.

The control unit can be produced operative to setting sequences for varying visible light color and intensity treating the eye retina and intensity of transscleral infrared laser radiation treating ciliary muscle of the eye.

### BRIEF DESCRIPTION OF THE DRAWINGS

FIG. 1 and FIG. 2 present diagrams of devices for therapy applied to ophthalmology.

### MODES FOR CARRYING OUT THE INVENTION

Firstly, the eye accommodation recovery e.g. the eye ability to clearly resolve distant and near objects is related to recovery of the eye muscle (accommodation apparatus) function, especially the ciliary muscle. The essence of the methods suggested for the eye accommodation recovery comprises a combination of transscleral effect on the ciliary muscle and treatment of accommodation apparatus via direct treating retina by visible light. Transscleral treatment of ciliary muscle by pulse infrared laser radiation intensifies blood circulation in the eye tissues. Treating retina can be a stimulus for the accommodation apparatus operation or promote eye muscle relaxation, wherein controlling the accommodation apparatus state and using increased blood circulation for accommodation recovery. Besides a combination of two different treatment ways for the accommodation apparatus, methods one and two for accommodation recovery suggest a coordination of these treatments.

Method one comprises retina treatment via varying color and intensity of irradiating light. The example of the method implementation below comprises the following three stages. Stage one (5-10 s long)—green light treats retina and relaxes (calms) the accommodation apparatus. Stage two (10-15 s long)—the treatment by green light is supplemented with pulse infrared laser radiation treatment gradually increasing intensity and, hence, increasing blood circulation in eye muscle vessels. Stage three (15-20 s long)—infrared laser radiation intensity is decreased and color is regularly changed to red, hence, stimulating the accommodation apparatus. In addition, for increasing the treatment intensity of the accommodation apparatus, at the stage three the light source is approached to or removed from the eye. Thereafter, the steps are repeated.

The method two comprises visible light treating the greater area of eye retina regularly or continuously and visible light color altering. Anyway, a part of visible light is used for fixing the sight direction during transscleral effect of pulse infrared laser radiation. An example below presenting implementation of the method comprises the following three stages. Stage one (5-10 s long)—a greater part of fixation field of the eye is treated by green light, hence, eye muscles calm and relax. Stage two (10-15 s long)—a part or the whole green light is collimated for fixing the sight direction simultaneously invoking pulse infrared laser radiation, wherein increasing blood circulation in eye muscle vessels. Stage three (15-20 s long)—regular, 0.5-0.7 s period, altering light color to red together with dispersed component, wherein stimulating the accommodation apparatus (training it). Thereafter, the steps are repeated.

Said examples the methods can be performed using corresponding devices, described below.

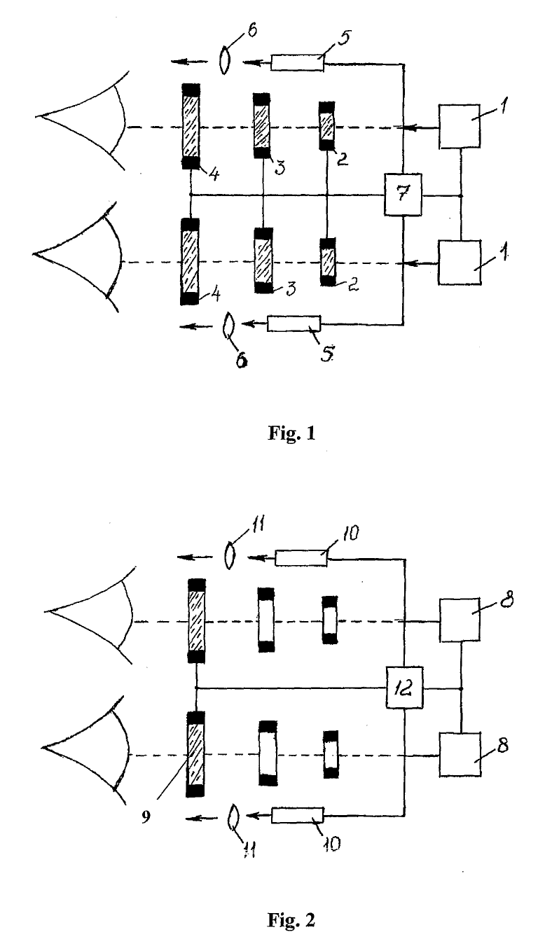

In accordance with the diagram in FIG. 1, the alternative one of said device for therapy in ophthalmology comprises visible radiation sources (1) for left and right eye, respectively, controllable radiation dispersing components (2), (3) and (4), lasers the sources of pulse infrared radiation (5), a lens systems (6) and a control unit (7). The control unit (7) is connected to all said components (except for lens systems (6). Hence, the changes of visible light source (1) parameters—color and radiation intensity, are synchronized with lasers (5) radiation intensity.

Tricolor diodes can be operative as visible light sources (1), for example, full-color light diode LF59EMBGMBW (Kingbright). Controllable radiation dispersion components (2), (3) and (4) can be produced as described, for example, in RU Patent 2,141,683, JP Patent 60,221,729 and in the international application PCT/DE2002/02593 (WO 03/009058). These components possess at least two states: transparent in one state permeating light freely, and completely dispersing light in another state.

Tandem controllable components for radiation dispersion (2), (3) and (4) provide for an effect of drawing closer or removing the visible light source. If the component (2) disperses light and units (3) and (4) are transparent, the light source resolved by the eye locates at the ultimate distance from it. If then the module (7) orders the component (3) to disperse light and components (2) and (4) remain transparent, the light source resolved by the eye will be drawn closer to it. Hence, naturally, the angular dimension of the visible light source increases, defined by components (2) and (3) dimensions. Accordingly, if then component (4) controlled by module (7) disperses light, and component (2) and (3) remain transparent, the light source resolved by the eye will maximally approach it.

In accordance with the diagram in FIG. 2, the alternative two of the device for therapy in ophthalmology comprises for left and right eye correspondingly the visible light sources (8), controllable components for radiation dispersion (9), lasers—the sources of pulse infrared radiation (10), lens systems (11), and a control unit (12). The control unit (12) is connected to all said components (except for lens systems (11). Hence, color variations of visible light sources (8), the state of controllable dispersion component (9) and laser (10) irradiation intensities are synchronized.

Tricolor diodes can be operative as visible light sources (1), for example, full-color light diode LF59EMBGMBW (Kingbright). Controllable radiation dispersion components (2), (3) and (4) can be produced as described, for example, in RU Patent 2,141,683, JP Patent 60,221,729 and in the international application PCT/DE2002/02593 (WO 03/009058). These components possess two or more states: once they are transparent and transmit light freely; in another state the completely disperse light. Others are intermediate states producing partly dispersed light.

The controllable light dispersion components (9) are located in output pupils of visible light formers. Hence, maximal “exposure” of the eye retina to light dispersed by the component (9) is provided. Since the visible light formers are required for laser (10) operation, two operation modes of the components (9) are provided. The mode one comprises regular light dispersion during short time periods (0.5 s or shorter) e.g. the periods having no effect on the eye orientation. The mode two represents gradual partial dispersion of radiation on components (9). In this mode, the eye will receive dispersed and collimated radiation for the source (8), simultaneously.

What is claimed is:

1. A method for recovery of accommodation of the eye, comprising transscleral effect of pulse infrared laser radiation on ciliary muscle using visible light for fixing the sight direction, characterized in that the light color used for fixing the sight direction is altered treating the eye retina and, hence, controlling the state of the accommodation apparatus of said eye, and the intensity of pulse infrared laser radiation treating the ciliary muscle is varied simultaneously.

2. The method of claim 1, characterized in that the light color is altered smoothly along the visible spectrum and at some regions of the visible spectrum the variation rate and regularity are selected individually.

3. The method of claim 1, characterized in that in addition the visible light source is approached to or removed from the eye varying visible angular dimension of the light source.

4. A method for recovery of accommodation of the eye, comprising transscleral effect of pulse infrared laser radiation on ciliary muscle using visible light for fixing the sight direction, characterized in that a part of visible light used for fixing the sight direction is dispersed in the vicinity of the eye pupil, providing for the effect of dispersed radiation on the greater area of retina, and the visible light color is altered simultaneously, wherein controlling the state of the accommodation apparatus of the eye.

5. The method of claim 4, characterized in that radiation used for fixing the sight direction id regularly dispersed.

6. The method of claim 5, characterized in that transscleral infrared laser radiation regularly treats the ciliary muscle simultaneously with dispersed radiation treating the eye retina.

7. The method of claim 5, characterized in that dispersed light color is altered in relation to light used for fixing the sight direction.

8. The method of claim 4, characterized in that a part of radiation treating the eye retina id continuously dispersed.

9. The method of claim 8, characterized in that the ratio of dispersed and collimated components of radiation treating the eye retina is regularly changed.

10. A device for therapy applied to ophthalmology, comprising at least one module for transscleral pulse infrared laser radiation treating the ciliary muscle and fixing the sight direction, comprising a former of pulse infrared laser radiation and a former of visible light, characterized in that additionally comprises a control unit connected to the former of visible light operative to alter color and intensity of light, and the former of pulse infrared laser radiation synchronizing color and intensity alterations of visible light treating the eye retina and alterations of transscleral laser radiation treating the ciliary muscle of the eye.

11. The device of claim 10, characterized in that it comprises the second module for transscleral pulse infrared laser radiation treating the ciliary muscle of the eye and fixing the sight direction, comprising a former of pulse infrared laser radiation and a former of visible light operative to alter color and intensity of the light, and the control unit is connected to formers of visible light and formers of pulse infrared laser radiation of both modules.

12. The device of claim 11, characterized in that the control unit is operative to set sequences of color and intensity alteration of visible light treating the eye retina and alterations of transscleral laser radiation intensity treating the ciliary muscle of the eye.

13. The device of claim 10, characterized in that the former of visible light is operative to change the angular dimension of the radiation source, and the control unit is operative to synchronize changes of angular dimension of the light source with color and intensity alterations of said light.

14. The device of claim 13, characterized in that the former of visible radiation comprises a tandem of several diaphragms with controllable components for dispersing visible light.

15. A device for therapy applied to ophthalmology, comprising at least one module producing transscleral pulse infrared laser radiation treating the ciliary muscle of the eye and fixing the sight direction, comprising a former of pulse infrared laser radiation and a former of visible light, characterized in that additionally comprising a control unit, said former of visible light operative to alter color of light comprises a component with controllable dispersion of visible light set at the output pupil of the former, the control unit is connected to the former of visible light and the former of pulse infrared laser radiation operative to synchronize color and dispersion alterations of visible light treating the ciliary muscle of the eye and changes of transscleral laser radiation intensity treating the ciliary muscle of the eye.

16. The device of claim 15, characterized in that comprising the second module for transscleral pulse infrared laser radiation treating the ciliary muscle of the eye and fixing the sight direction, comprising a former of pulse infrared laser radiation and a former of visible light operative to alter the light color and comprising a component with controllable dispersion of visible light, the control unit is connected to the formers of visible light and the formers of pulse infrared laser radiation of both modules.

17. The device of claim 15, characterized in that the control unit is operative to set sequences of color and dispersion alterations of visible light treating eye retina and changes of transscleral infrared laser radiation intensity treating the ciliary muscle of the eye.
